(19) United States
(12) Patent Application Publication
(10) Pub. No.: US 2024/0335665 A1
(43) Pub. Date: Oct. 10, 2024

(54) INTERLEAVED NEURAL STIMULATION

(71) Applicants: NEURINNOV, Montpellier (FR); INRIA-INSTITUT NATIONAL DE RECHERCHE EN INFORMATIQUE ET EN AUTOMATIQUE, Le Chesnay (FR); UNIVERSITÉ DE MONTPELLIER, Montpellier (FR)

(72) Inventors: David GUIRAUD, Montpellier (FR); David ANDREU, Montpellier (FR)

(73) Assignees: NEURINNOV, Montpellier (FR); INRIA - INSTITUT NATIONAL DE RECHERCHE EN INFORMATIQUE ET EN AUTOMATIQUE, Le Chesnay (FR); UNIVERSITÉ DE MONTPELLIER, Montpellier (FR)

(21) Appl. No.: 18/293,096

(22) PCT Filed: Jul. 29, 2022
(86) PCT No.: PCT/FR2022/051524

§ 371 Date: Jan. 29, 2024

(30) Foreign Application Priority Data
Jul. 30, 2021 (FR) FR2108317

(51) Int. Cl. A61N 1/36; A61N 1/05

(52) U.S. Cl. CPC A61N 1/36157; A61N 1/0556; A61N 1/36171; A61N 1/36175; A61N 1/36178

(57) ABSTRACT

A neural stimulation device configured to apply neural activations including at least two interleaved sequences of current pulses, and a neurostimulation method. The device includes at least one neural electrode including at least two electrical contacts and intended to be implanted on at least one nerve; a current pulse generator connected to the at least one neural electrode via a current distributor; a storage element configured to contain a library of sequences, each sequence including a time series of current pulses associated with a configuration of electrical contacts; and a sequencer configured to apply neural activation to the at least one neural electrode, the neural activation including at least two interleaved sequences.

### FIELD OF INVENTION

The present invention relates to neural stimulators, in particular stimulators intended for restoring the movement of paralyzed limbs. These stimulators are intended to activate one or more muscles by exciting a nerve by means of an electrical stimulus.

### BACKGROUND OF INVENTION

The proportion of people with tetraplegia following spinal or cervical trauma is constantly growing. Improving the quality of life of these people is a challenge, and restoring grip ability is a priority, as hand use is a key factor in the quality of daily life. Different approaches have been developed to restore the functions of the hand, wrist and forearm, which contribute to the gripping mechanism. Surgical methods—musculotendinous transfers or nerve transplants—are possible in cases where a sufficient number of muscles are still voluntarily controlled by the quadriplegic person. Alternatively, it is possible to use electrical stimulation methods, either applied directly to the muscles—which requires numerous and invasive devices—or applied to intact nerves downstream of the lesion. This last method consists of placing electrodes on the nerves providing the innervation of the muscles controlling the hand and the wrist, in particular: the median nerve, the ulnar (or cubital) nerve and the superficial branch of the radial nerve. The two approaches, muscle and neural stimulation, can be combined.

It is now known to selectively stimulate the median and radial nerves in order to restore elementary movements such as the subterminal opposition grip (clamp), the palmar (spherical) penta-digital grip or the full hand grip for examplesee Selective neural electrical stimulation restores hand and forearm movements in individuals with completed tetraplegia; Tigra et al. Journal of NeuroEngineering and Rehabilitation (2020) 17:66; DOI: 10.1186/s12984-020-00676-4.

However, these stimulation methods remain incomplete because they do not restore the prehension movements in all their complexity. The present invention aims at overcoming these limits by proposing a stimulation device applying more complex, in particular neural, stimulations.

### SUMMARY

The invention therefore relates to a neural stimulation device comprising
at least one neural electrode comprising at least two electrical contacts and intended to be implanted on at least one nerve;
a current pulse generator connected to the at least one neural electrode via a current distributor;
a storage element configured to contain a library of sequences, each sequence comprising a time series of current pulses associated with a configuration of electrical contacts; and
a sequencer configured to apply neural activation to the at least one neural electrode, said neural activation comprising at least two interleaved sequences.

In one embodiment, the sequences to be interleaved comprise the same number of intervals, these intervals having the same duration.

In one embodiment, the neural stimulation device further includes a controller configured to drive a succession of neural activations. In particular, the controller configures the sequencer then activates the sequences to be interleaved.

In one embodiment, the at least one neural electrode includes a plurality of electrical contact configurations.

In one embodiment, the current pulses are load-balanced biphasic stimuli. Preferably, the first phase of the load-balanced biphasic stimuli has an amplitude between 10 μA and 6000 μA and a duration between 5 μs and 1 ms.

In one embodiment, the sequences comprise a succession of 5 to 100 current pulses, said pulses being repeated at a frequency between 1 Hz and 1 kHz.

In one embodiment, the sequence library contains personalized sequences for a patient.

The invention also relates to a neurostimulation method comprising
the selection of at least two sequences from a sequence library, each sequence comprising a time series of current pulses associated with a configuration of electrical contacts; and
the application of a neural activation to at least one neural electrode comprising at least two electrical contacts and intended to be implanted on at least one nerve, said neural activation comprising the interleaving of the at least two sequences and the addressing of the pulses of current from the at least two sequences to the at least two electrical contacts by a current distributor.

In one embodiment, the neurostimulation method further includes repeating the application of neural activation.

The invention finally relates to a neural activation comprising
interleaving of at least two sequences chosen from a sequence library, each sequence comprising a time series of current pulses associated with a current distribution between several electrical contacts; and
addressing the current pulses of the at least two sequences to the at least two electrical contacts of a neural electrode intended to be implanted on a nerve.

### Definitions

In the present invention, the terms below are defined as follows:

“Electrical contact” relates to a surface of a neural electrode, this surface being in contact with the nerve to be stimulated and electrically connected to the neural stimulation device. A neural electrode may include several electrical contacts distributed over different rings.

“Electrical contact configuration” refers to a configuration in which each electrical contact of a neural electrode is associated with an anode “a” function, cathode “c” function or is not connected “-”.

“Stimulation configuration” concerns a configuration in which the amplitudes of the current pulses which are sent to each contact are defined, for a given electrical contact configuration. The distribution of the amplitudes of the current pulses is defined by a current distributor.

“Neural electrode” concerns an electrode intended to be implanted in contact with a nerve in order to apply electrical stimuli.

“Interleaving” concerns the temporal superimposition of two—or more—series of electrical signals. Thus, two—or more—sequences of current pulses are applied alternately such that one sequence begins before another sequence ends. The sequences of current pulses can also be applied in a composed way, if the pulses are sent simultaneously on different electrical contact configurations—i.e., in the absence of configuration conflict which would see the same electrical contact implemented with different functions (anode/cathode or different amplitudes) for the two configurations—of the same neural electrode.

“Current pulse” refers to a brief electrical signal, characterized by its shape, amplitude and duration. The current pulse is typically rectangular, but can adopt any other shape-triangular, asymmetrical, biphasic . . . depending on the neural stimulation needs. The pulse amplitude is typically between 10 μA and 10 mA. The duration of the pulse is typically between 5 μs and 1 ms. In addition, the current pulse can be positive-injection of electrical charges—or negative—withdrawal of electrical charges.

“Biphasic stimulus” concerns a stimulus comprising two phases, separated by an interstimulus interval aimed at maintaining the induced pre-polarization by the first phase of the biphasic stimulus. The interstimulus interval is typically between 25 μs and 150 μs.

“Load-balanced biphasic stimulus” relates to a biphasic stimulus for which the quantity of charges implemented during the first phase—charges injected—is equal to the quantity of charges implemented during the second phase—charges removed—the signs of charges being however opposed in the two phases. In total, no net charge is injected or removed by a charge-balanced biphasic stimulus. The two phases can have different shapes, amplitudes and durations provided that the quantities of charges are identical.

### DETAILED DESCRIPTION

The present invention relates to a neural stimulation device.

This device comprises at least one neural electrode intended to be implanted on at least one nerve. In the particular case of neural stimulation for the restoration of grip, the neural electrode is implanted on one of the following nerves: the median nerve, the ulnar (or cubital) nerve or the radial nerve. The neural electrode has at least two electrical contacts.

The neural electrode can be chosen from flat matrix electrodes, intended to be placed on the nerve; intra fascicular electrodes, intended to be placed inside the nerve; or cuff electrodes intended to be placed around the nerve. Cuff electrodes are particularly suitable because they make it possible to regularly distribute several electrical contacts all around the nerve. Advantageously, the electrical contacts are distributed on the cuff electrode over three rings: two outer rings on which a single electrical contact completely surrounds the nerve and an inner ring on which several contacts—typically four to ten—are distributed regularly. The self-adjusting cuff electrodes allow the contacts of the inner ring to be distributed over the nerve by adapting to its diameter.

The neural stimulation device includes a current pulse generator. This generator is connected to the neural electrode via a current distributor. The current distributor allows to distribute the injection or withdrawal of electrical charges associated with the current pulse between the different electrical contacts of the neural electrode. This distribution is subsequently called an electrical contact configuration: it contains both the selected electrical contacts of the neural electrode and the current distribution between these contacts. Preferably, the neural electrode comprises a plurality of electrical contact configurations. Thus, by distributing the current pulses over different areas of the nerve-on the surface of the nerve for a cuff electrode—it is possible to stimulate the fascicles of the nerve in a different way, each fascicle contributing to the control of at least one muscle.

Preferably, the current distributor can amplify the electrical pulse for each electrical contact. Thus, the current pulse generator defines the shape, duration and reference amplitude of the pulse when the current distributor defines the real amplitude-after amplification—and the distribution of the real amplitudes on the different electrical contacts. This weighted distribution is hereafter called a stimulation configuration.

In one embodiment, the current pulses are load-balanced biphasic stimuli. In this case, the shape of the pulse includes two phases: the first phase—called stimulation—is an injection of electrical charge and the second phase—called balancing—is a withdrawal of electrical charges. The pulse is load-balanced when the quantity of electrical charge injected is equal to the quantity of electrical charge removed, generally leading to avoiding the accumulation of electrical charges on the stimulated nerve and/or on the electrical contacts between the neural electrode and the nerve stimulated. The first phase of a load-balanced two-phase stimulus has an amplitude between 10 μA and 6 mA and a duration between 5 μs and 1 ms. The second phase is then defined in amplitude and duration due to load balance. The two phases can be separated by an interstimulus interval. More generally, current pulses can be multiphasic stimuli-several phases of charge injection or charge withdrawal—as long as the quantities of charges injected and charges removed remain equal.

For example, the current generator can define a load-balanced biphasic stimulus comprising a first rectangular phase of reference amplitude 100 μA and duration 20 μs and a second rectangular phase of reference amplitude 50 μA and duration 40 μs. Then the current distributor applies this load-balanced biphasic stimulus between the anodes: the two outer rings and contacts 1 and 3 of the inner ring and the cathode: contact 2 of the inner ring—contacts 5 to 8 being not connected in this configuration of electrical contacts—of a cuff electrode in a configuration of electrical contacts TTR. Additionally, the pulse is amplified by a factor of 3 on the outer rings and on contacts 1 and 3 and amplified by a factor −12 on contact 2—leading to a balanced amplification—and defining a configuration stimulation.

The neural stimulation device further includes a storage element configured to contain a library of sequences. This storage element may be a memory included in the neural stimulation device or an external storage element in wireless communication with the neural stimulation device. A sequence is a time series of pulses, in which a pulse is repeated many times. This repetition can be strictly periodic: the same pulse repeated at a given frequency, or modulated. The modulation can relate to the frequency—the repetition period of the pulses is variable during the sequence—the amplitude—the amplitude of each pulse varies during the sequence—and/or the duration of the pulse. Additionally, the sequence is associated with a configuration of electrical contacts defining how the current pulses of the sequence should be distributed across the nerve. Each sequence in this library is associated with the stimulation of one or more fascicles of the same nerve, the effect of which is to contract target muscles leading to a complex movement.

Finally, the neural stimulation device includes a sequencer configured to apply neural activation to the neural electrode. This neural activation comprises at least two interleaved sequences. By interleaving, it is meant here that the time series of current pulses applied to the neural electrode is the superposition of the time series of current pulses of each sequence. However, the neural stimulation device can only implement one stimulation configuration at a time, so the interleaving must lead to applying the current pulse of one sequence between two current pulses of the other sequence (or other sequences). In particular, the application of two interleaved sequences corresponds to defining a succession of stimulation configurations on the neural electrode, the stimulation configurations being associated with two different sequences. In certain embodiments, the interleaving may concern three sequences, four sequences or more.

To interleave two sequences, the sequencer therefore receives the information from each of the sequences-signal shape, stimulation configuration and signal repetition interval/number—and applies it taking into account the following constraints:
the durations of the intervals of the sequences are identical, which allows to insert the signal of one sequence between two signals of another sequence,
stimulation configurations are compatible, or can be modified between applications of signals from different sequences.

In the case where the muscles to be used for a complex movement act simultaneously, the sequences to be interleaved include the same number of intervals, these intervals having the same duration. By starting the sequences at the same time, the effects of the neural stimulations are induced simultaneously, leading for example to simultaneously activate the targeted muscles. Alternatively, if muscles are to be used in a specific order, a first sequence can begin and a second sequence be interleaved later into the first. The first sequence may end before the second sequence. This principle also applies to three, four or more sequences.

A neural activation therefore includes several sequences whose signals are applied alternately. A first sequence begins by applying a succession of electrical signals separated by neutral times-absence of signal-, then a second sequence is interspersed to apply electrical signals during the neutral times of the first sequence. The second sequence therefore begins before the first sequence ends.

The interleaving of sequences can also rely on the modulation of the neutral times of each sequence, to better manage the constraint of intercalation of signals. For example, the sequencer can reduce the neutral times of the two sequences by half, so that the total neutral time between two successive signals of the same sequence is preserved.

By interleaving two sequences, it is possible to stimulate two simultaneous and/or cooperative responses with a single nerve. In the case of muscular responses, this leads to a complex movement that is synergistic (cooperation in movement) or antagonistic (stabilization effect or increase in joint stiffness) for example.

In one embodiment, the neural stimulation device further comprises a controller configured to drive a succession of neural activations. This allows to create a succession of complex movements whose sequence leads to the performance of a motor function, for example opening the hand followed by a palmar grip, thus restoring grip. Alternatively, the succession of neural activations—at a determined frequency—can simply lead to the maintenance of one—or more—muscular contraction.

In one embodiment, the controller can control the sequencer in order to interleave the sequences. In this case, the controller configures the sequencer then activates the sequences and it is at the controller level that the application constraints of the sequences are verified. This embodiment is advantageous because it minimizes information transfers between the controller and the electrical signal generator. In fact, the controller configures the sequencer, and the sequencer relies on the sequence library to generate the signals. Thus, the controller that manages the high-level neural stimulations, depending on the expected muscular effects, is decoupled from the very precise generation of the signals—with temporal precisions that are not affected by the transmission of data from the controller to the generation of the signals.

In one embodiment, the sequences comprise a succession of 5 to 100 current pulses, preferably 10 to 100 current pulses. These current pulses are repeated at a frequency between 1 Hz and 1 kHz, this frequency possibly being modulated. In the case of efferent stimulations (for motor purposes), the current pulses are repeated preferably at a frequency between 1 Hz and 250 Hz, typically between 20 Hz and 100 Hz. The duration of a complete sequence is very variable, depending on the task to be carried out. In particular, to maintain a muscle contraction, the sequence must last the entire time the muscle contraction is maintained. Preferably, a complete sequence lasts at least 0.5 s and includes at least 10 pulses.

In one embodiment, the pulse generator is designed to very precisely control the charges injected or removed at the neural electrodes. For this, the pulse generator can rely on a symmetrical design comprising anode current sources and cathode current sources configured to copy a determined control current and so that the sum of the copied anode currents is equal to the sum of the copied cathode currents.

In one embodiment, the sequence library contains sequences personalized for a patient. This is because the stimulation configurations and current pulses that effectively stimulate a patient vary from patient to patient. It is therefore necessary to define personalized sequences during a learning process. This learning process can take place as follows.

After the neural stimulation device is set up by implanting a neural electrode on a patient's nerve, template sequences are applied. These template sequences are defined by the following parameters: shape, amplitude and duration of the current pulse; stimulation configuration-electrical contacts used, and associated amplification-; number of current pulses and frequency of repetition of current pulses-defining an interval between two successive signals. A succession of sequences in which the parameters are varied one by one allows to identify the thresholds at which the patient's muscles act, allowing to construct a recruitment curve for each of them. FIG. 2 illustrates for a patient, with a cuff electrode implanted around the median nerve, for electrical contact configurations of the TLR, STR and TTR type, the cathode of which is defined successively on each of the eight electrical contacts of the inner ring—each sector corresponds to one of the electrical contacts taken as the cathode in this graphic representation—, for stimulation sequences of 15 repetitions at 25 Hz of a balanced biphasic stimulus of 150 μs duration, the real amplitudes being increasing—after amplification by the sequencer—of the impulse selectively stimulating the APB, LDS, LPL, PT or PCR muscles. We see for example that the configuration of electrical contacts TLR allows to recruit with the lowest intensity (therefore first) the PPL muscle for positions 6-7-8 of the cathode, the other contacts allowing to preferentially activate other muscles. In addition, each contact and each type of configuration (TLR, STR, TTR) allows to obtain a succession of muscular activation, and therefore a different muscular synergy. It is thus possible, for example, to obtain a flexion of the fingers then of the thumb to obtain a clamp by stimulating only the median nerve with a configuration preferentially recruiting the PDS and PPL muscles.

Thus, for each muscle taken individually, and more broadly each sequence of muscular contractions linked to the increase in intensity, it is possible to define the sequence which allows to activate a synergistic and functional muscular contraction, for a given patient. Then, based on the personalized sequences, it is possible to interleave at least two sequences to apply neural stimulation and cause a complex compound movement, for example hand opening and wrist extension simultaneously, and then set a succession of neural activations to restore a motor function, for example opening the hand then closing in clamp mode.

In one embodiment, the entire stimulation device is implantable and further comprises an element for wireless communication. Thus, a patient can be equipped with the stimulation device during a surgical procedure. Then, commands can be transmitted to the sequencer and/or to the stimulation device controller by a control device positioned in contact with the patient-held by an armband for example—and controlled by the patient himself. The patient can activate the control device vocally, by voluntary activation of muscles equipped with electromyographic detectors, by performing stereotyped voluntary movements detected by inertial sensors or by a brain/machine interface, for example.

In one embodiment, the stimulation device comprises at least two neural electrodes, intended to be implanted on at least two different nerves. Thus, complex stimulations implementing muscles activated by the various nerves equipped with neural electrodes can be carried out, for example to obtain the closure of the hand in the form of a clamp (stimulation on the median nerve) and the balance of the wrist by its antagonistic extension (stimulation on the radial nerve).

The invention also relates to a method of neurostimulation, in which the characteristics described above for the neural stimulation device apply.

This method includes a step of selecting from a sequence library at least two sequences, each comprising a time series of current pulses associated with a configuration of electrical contacts. In a second step, neural activation is applied to at least one neural electrode comprising at least two electrical contacts and intended to be implanted on at least one nerve. This neural activation results from the interleaving of the at least two sequences and the addressing of current pulses from the at least two sequences to the at least two electrical contacts by a current distributor.

In one embodiment, the neurostimulation method further includes repeating the application of neural activation. Thus, several neural activations, each resulting from the interleaving of at least two sequences, are applied successively.

Finally, the invention relates to neural activation, which is a particular form of electrical signal. The characteristics described above for the neural stimulation device apply here.

This neural activation comprises on the one hand the interleaving of at least two sequences chosen from a library of sequences, each sequence comprising a time series of current pulses associated with a distribution of current between several electrical contacts. It also comprises the addressing of the current pulses of the at least two sequences to the at least two electrical contacts of a neural electrode intended to be implanted on a nerve.

In other words, a neural activation is an electrical signal associated with a configuration of electrical contacts and which allows to obtain a response, for example muscular. A neural activation is an elementary instruction which can then be inserted into a sequence of instructions allowing to carry out functions, in particular motor functions.

In one embodiment, the addressing of the current pulses includes the electrical contact patterns of the at least one neural electrode.

In one embodiment, the current pulses are load-balanced biphasic stimuli, preferably with the first phase having an amplitude between 10 μA and 6000 μA and a duration between 5 μs and 1 ms.

In one embodiment, the sequences comprise a succession of 1 to 100 current pulses. These current pulses are repeated at a frequency between 1 Hz and 1 kHz, this frequency possibly being modulated. In the case of efferent stimulations (for motor purposes), the current pulses are repeated preferably at a frequency between 1 Hz and 250 Hz, typically between 20 Hz and 100 Hz. The duration of a complete sequence is very variable, depending on the task to be performed. In particular, to maintain a muscle contraction, the sequence must last the entire time the muscle contraction is maintained. Preferably, a complete sequence lasts at least 0.5 s and includes at least 10 pulses.

### BRIEF DESCRIPTION OF THE DRAWINGS

FIG. 1 is a representation of a cuff-type neural electrode. This electrode (top left) includes three rings A, B and C. Each ring has four electrical contacts marked 1, 2, 3 and 4. On the outer rings A and C, the four contacts are connected so as to form a single electrical potential all around the nerve. On inner ring B, the four contacts are separate and can be connected differently. The anode connections are denoted “a”, the cathodic connection is denoted “c” and the unconnected electrical contacts are denoted “-”. In the TLR electrical contact configuration, the outer rings form two anodes and the inner ring has a cathode on one of its electrical contacts. In the TTR electrical contact configuration, the TLR electrical contact configuration is completed by two anodes located on the inner ring, immediately adjacent to the cathode. In the STR electrical contact configuration, the TLR electrical contact configuration is completed by an anode located on the inner ring, diametrically opposite the cathode. Finally, in the TT electrical contact configuration, only one ring is used (here, the inner ring noted B, but this could be an outer ring A or C) and has a cathode on one of its electrical contacts and two anodes immediately adjacent to the cathode.

FIG. 2 shows the order of muscle recruitment for a patient in various electrical contact configurations; left dial: TLR; central dial: STR; right dial: TTR. Each dial contains eight sectors corresponding to the eight electrical contacts of the inner ring of the cuff-type neural electrode. Each sector represents the order of successive recruitment of each muscle (as soon as it exceeds 10% of its recruitment) when the electrical contact of the interior ring corresponding to the sector is used as cathode.

FIG. 3 depicts neural activation resulting from the interleaving of two sequences, each sequence comprising current pulses in the form of balanced biphasic stimuli.

### ILLUSTRATIVE EMBODIMENTS OF THE INVENTION

The present invention will be better understood on reading the following examples which illustrate the invention in a non-limiting manner.

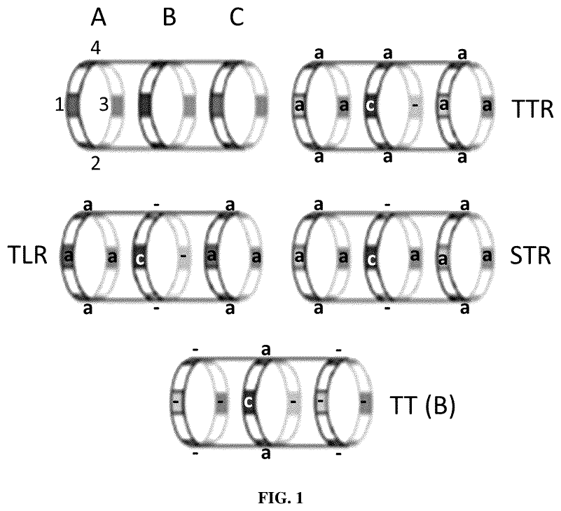

A patient suffering from quadriplegia underwent surgery to implant two cuff electrodes, one on the median nerve and the other on the radial nerve. The cuff electrodes had the structure described in FIG. 1, with two outer rings and a central ring having 8 electrical contacts for the median nerve and 6 electrical contacts for the radial nerve. The electrical contact configurations used are the TTR (transverse three-pole), TLR (longitudinal three-pole) and STR (steering current) configurations, shown in FIG. 1.

In order to create a personalized sequence library for this patient; different stimulation configurations-contact configuration, pulse amplitudes and sequence-were applied and the muscular responses measured via electromyographic measuring devices, by image analysis of the movements caused or by measuring the forces exerted by the stimulated muscles. These measures have allowed to define recruitment curves for each muscle. Thus, the responses induced by the median nerve were determined:
flexor carpi radialis (FCR) responsible for wrist flexion,
pronator teres (PT) responsible for pronation of the forearm and wrist,
flexor digitorum superficialis (FDS) responsible for flexion of the fingers (except the thumb)
flexor pollicis longus (FPL) responsible for flexion of the thumb, and,
abductor pollicis brevis (APB) responsible for thumb abduction.

Furthermore, the responses induced by the radial nerve were determined:
extensor carpi radialis (ECR) responsible for wrist extension were determined,
extensor pollicis longus (EPL) responsible for thumb extension, and,
extensor digitorum communis (EDC) responsible for extending the fingers.

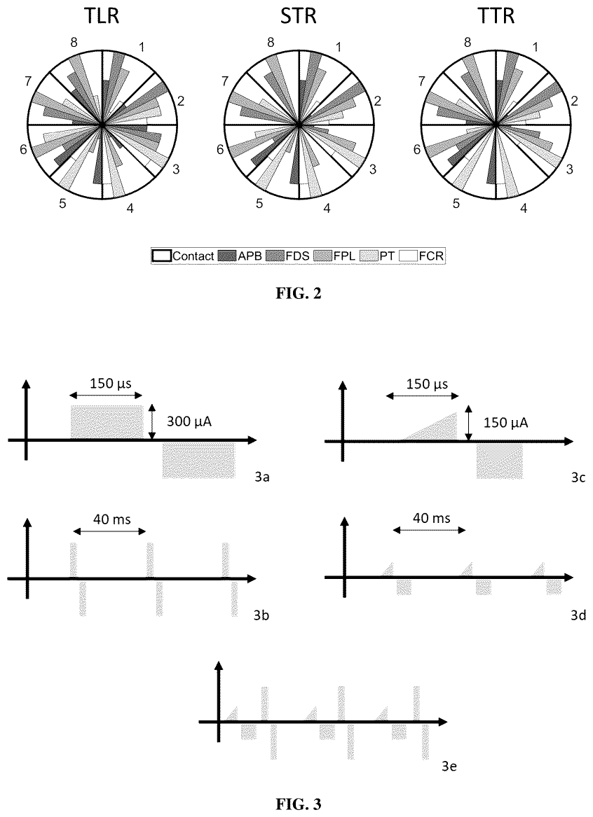

FIG. 2 represents the succession of muscle recruitments when the stimulation intensity increases for a configuration of electrical contacts-forming a stimulation configuration-allowing to recruit each muscle: recruitment of APB, FDS, FPL, PT and FCR in the configurations of TLR, STR and TTR contacts. These graphs associated with the recruitment curves of each muscle make it possible to identify, for a given contact configuration, the levels from which said muscles are recruited and the order in which these said muscles are activated.

It is then possible to define personalized sequences for the patient, these sequences allowing to selectively activate a muscle or several muscles simultaneously.

FIG. 3 represents in a simplified manner two sequences from the library. A load-balanced and symmetrical biphasic stimulus-FIG. 3-a, first phase of amplitude 300 μA and duration 150 μs and identical second phase, but of opposite sign, after an interstimulus interval of 25 μs—is repeated at a frequency of 25 Hz—period of 40 ms—to form a first sequence-FIG. 3-b—of 15 pulses. A load-balanced biphasic stimulus—FIG. 3-c, first phase of linearly increasing amplitude up to 150 μA and duration 150 μs and second phase of amplitude 150 μA and duration 75 μs but of opposite sign, after a time of 25 μs interstimulation—is repeated at a frequency of 25 Hz-period of 40 ms—to form a second sequence-FIG. 3-d—of 15 pulses. Each sequence allows to successively recruit one to several patient muscles, when associated with a configuration of contacts and a particular amplification, defined by the corresponding recruitment curve. It is therefore a question of inducing synergies of different muscular actions. These synergies can then be combined by interleaving the sequences on one or more nerves.

In order to coordinate multiple muscle movements and obtain a motor function, the two sequences are interleaved, as illustrated in FIG. 3-e. It should be noted that successive pulses are applied by different contact configurations, but after each pulse, no net charge has been injected into the patient, due to the load-balanced nature of the pulses.

The application of neural stimulation of these two interleaved sequences leads to complex movements that cannot be obtained by successive application of two sequences. Indeed, if the sequences are applied successively, the muscles activated by a sequence will relax when this sequence ends and will not be able to act in synergy with the muscles activated by the following sequence.

1-11. (canceled)

12. A neural stimulation device comprising:
at least one neural electrode comprising at least two electrical contacts and intended to be implanted on at least one nerve;
a current pulse generator connected to the at least one neural electrode via a current distributor;
a storage element configured to contain a library of sequences, each sequence comprising a time series of current pulses associated with a configuration of electrical contacts; and
a sequencer configured to apply neural activation to the at least one neural electrode, said neural activation comprising at least two interleaved sequences.

13. The neural stimulation device according to claim 12, wherein the sequences are interleaved alternately such that one of said sequences begins before another of said sequences ends.

14. The neural stimulation device according to claim 12, wherein the sequences to be interleaved comprise the same number of intervals, these intervals having the same duration.

15. The neural stimulation device according to claim 12, further comprising a controller configured to drive a succession of neural activations.

16. The neural stimulation device according to claim 15, wherein the controller configures the sequencer and then activates the sequences to be interleaved.

17. The neural stimulation device according to claim 12, wherein the at least one neural electrode comprises a plurality of electrical contact configurations.

18. The neural stimulation device according to claim 12, wherein the current pulses are load-balanced biphasic stimuli.

19. The neural stimulation device according to claim 18, wherein the first phase of the load-balanced biphasic stimuli has an amplitude of between 10 μA and 6000 μA and a duration of between 5 μs and 1 ms.

20. The neural stimulation device according to claim 12, in which the sequences comprise a succession of 5 to 100 current pulses, the said pulses being repeated at a frequency comprised between 1 Hz and 1 kHz.

21. The neural stimulation device according to claim 12, wherein the sequence library contains sequences personalized for a patient.

22. A neurostimulation method including:
the selection of at least two sequences from a sequence library, each sequence comprising a time series of current pulses associated with a configuration of electrical contacts; and
the application of neural activation to at least one neural electrode comprising at least two electrical contacts and intended to be implanted on at least one nerve, said neural activation comprising the interleaving of the at least two sequences and the addressing of the pulses of current from the at least two sequences to the at least two electrical contacts by a current distributor.

23. The neurostimulation method according to claim 22, wherein the sequences are interleaved by applying alternately such that one of said sequences begins before another of said sequences ends.

24. The neurostimulation method according to claim 22, further comprising repeating the application of neural activation.

25. A neural activation including:
interleaving of at least two sequences chosen from a sequence library, each sequence comprising a time series of current pulses associated with a current distribution between several electrical contacts; and
addressing the current pulses of the at least two sequences to the at least two electrical contacts of a neural electrode intended to be implanted on a nerve.

26. The neural activation according to claim 25, wherein the sequences are interleaved alternately such that one of said sequences begins before another of said sequences ends.
